(19) United States
(12) Patent Application Publication
(10) Pub. No.: US 2011/0223062 A1
(43) Pub. Date: Sep. 15, 2011

(54) AUTOMATIC ANALYSIS DEVICE

(76) Inventors: Yusuke Minemura, Hitachinaka (JP); Goro Yoshida, Hitachinaka (JP); Kazuhiro Tanaka, Hitachinaka (JP)

(21) Appl. No.: 13/130,376

(22) PCT Filed: Nov. 13, 2009
(86) PCT No.: PCT/JP2009/069332

§ 371 Date: Jun. 1, 2011

(30) Foreign Application Priority Data
Nov. 21, 2008 (JP) 2008-297820
Dec. 5, 2008 (JP) 2008-310384

(51) Int. Cl. G01N 33/00

(52) U.S. Cl. 422/67

(57) ABSTRACT

An automatic analysis device capable of reducing readers and suppressing production cost without lowering efficiency of read processing of code information attached to a reagent container is provided. The automatic analysis device includes one two-dimension code reader 21 reading code information of a two-dimension code label 14 attached to a reagent container 8 and a control device analyzing a sample to be a target of an analysis, the two-dimension code reader 21 computing position information of a position at which the two-dimension code label 14 is attached and outputting the position information with the read code information when reading the two-dimension code label 14 attached to the reagent container 8; and the control device determining an inner periphery and an outer periphery of a reagent cool box based on the position information and storing information related to a reagent with information of an installed place of the reagent container 8.

### TECHNICAL FIELD

The present invention relates to an automatic analysis device, more particularly, reading of information such as reagent identification information etc. attached to a reagent container to be used in an analysis.

### BACKGROUND ART

An automatic analysis device has means of attaching a barcode to a container which contains liquid such as reagent, sample, cleaning substance, etc. to identify the contained liquid, thereby installing a function of reducing workload of inputting information of the liquid on a keyboard by a device operator himself/herself.

In the initial period of installing the technology, barcodes were just memorizing identification information of liquid, but in these years, it is desired that a lot of information items such as analysis conditions are memorized in addition to identification information.

That is, while existing devices read analysis conditions memorized in another memory medium based on identification information of liquid and use the read analysis conditions, if analysis conditions can be also memorized in barcodes, the analysis is started without accessing the memory medium and it improves convenience.

Here, the size of barcodes is increased as the data capacity is increased. Therefore, for example, a two-dimension code capable of memorizing more information items in the same space such as that described in Japanese Patent Application Laid-Open Publication No. 8-94626 (Patent Document 1) has been getting popular.

More information items can be handled by using the two-dimension code than using barcodes, and thus spread of a device having high convenience is expected.

### PRIOR ART DOCUMENT

### Patent Document

Patent Document 1: Japanese Patent Application Laid-Open Publication No. 8-94626

### DISCLOSURE OF THE INVENTION

### Problems to be Solved by the Invention

Meanwhile, a reader for reading the two-dimension code is very expensive, and so there is a problem in cost for mounting many readers. For example, when there are two reagent discs which store reagent containers containing reagents and the reagent containers can be mounted at an inner periphery and an outer periphery of one disc, four readers are required at maximum.

Here, to have one reader for the inner periphery and outer periphery, it is necessary to move the reader itself for moving the field of view of the reader to the inner periphery and outer periphery and it leads to a cost increase as a mechanism for moving is required. Also, as a time for moving is added in a read time, the process efficiency is lowered.

Meanwhile, when attaching a label of code on a side surface of a reagent bottle having a relatively large area, one-dimension barcode, which is inexpensive, may be used but the reader is accordingly arranged at the side surface of the bottle, resulting in an increase in the device size by the reader size.

Accordingly, a preferred aim of the present invention is to provide an automatic analysis device capable of suppressing production cost by reducing readers without lowering read process efficiency of code information attached to a reagent container.

Also, to read information of reagent containers at a plurality of peripheries by one reader, it is necessary to identify which periphery the read information is from, the inner periphery or the outer periphery, and, when the reader is one, in consideration of quickly performing reagent registration, a method of identification by simultaneous reading has been desired than identifying by reading information items of the inner periphery and outer periphery one by one.

Another preferred aim of the present invention is to provide an automatic analysis device capable of reading information items of reagent containers of a plurality of peripheries by one reader and also identifying and reading information items of reagent containers per periphery in the plurality of peripheries by one reader at the same time.

The above and other preferred aims and novel characteristics of the present invention will be apparent from the description of the present specification and the accompanying drawings.

### Means for Solving the Problems

The typical ones of the inventions disclosed in the present application will be briefly described as follows.

More specifically, a brief description of a typical invention is that a code reader calculates, upon reading code information attached to a reagent container, position information of a position at which code information is attached, and outputs the position information together with read code information; and a control device determines, based on the code information and position information from the code reader, which periphery the code information of a reagent container installed to the reagent installing means of the periphery belongs to among the reagent installing means of the plurality of peripheries of the cool box, and stores information relating to the reagent together with information of an installed place of the reagent container, and saves information related to the reagent.

Also, a brief description of another typical invention is that a code reader reads, upon reading code information attached to a reagent container, code information items of different code types of reagent installing means of a plurality of peripheries of a reagent cool box per periphery, and outputs information of a code type together with the read code information; and a control device determines which periphery the code information of a reagent container installed to the reagent installing means of the periphery belongs to among the reagent installing means of the plurality of peripheries of the cool box, and stores information relating to the reagent together with information of an installed place of the reagent container.

### Effects of the Invention

The effects obtained by typical aspects of the present invention will be briefly described below.

More specifically, an effect obtained by a typical one of the invention is that readers can be reduced and a production cost can be suppressed without lowering read processing efficiency of code information attached to a reagent container.

### BRIEF DESCRIPTIONS OF THE DRAWINGS

FIG. 1 is a structure diagram illustrating an overall configuration of an automatic analysis device according to a first embodiment of the present invention;

FIG. 2 is a top view of a reagent cool box of the automatic analysis device according to the first embodiment of the present invention;

FIG. 3 is a diagram illustrating an arrangement of reagent containers of the automatic analysis device according to the first embodiment of the present invention;

FIG. 4 is a diagram illustrating an arrangement of a two-dimension code reader of the automatic analysis device according to the first embodiment of the present invention;

FIG. 5 is a diagram illustrating an outer appearance of the reagent container of the automatic analysis device according to the first embodiment of the present invention;

FIG. 6 is an explanatory diagram for describing a reading range of the two-dimension code reader of the automatic analysis device according to the first embodiment of the present invention;

FIG. 7 is an explanatory diagram for describing the reading range of the two-dimension code reader of the automatic analysis device according to the first embodiment of the present invention;

FIG. 8 is an explanatory diagram for describing a reading operation without having a coordinate computing function on the two-dimension code reader of the automatic analysis device according to the first embodiment of the present invention;

FIGS. 9A and 9B are explanatory diagrams for describing a reading operation without having a coordinate computing function on the two-dimension code reader of the automatic analysis device according to the first embodiment of the present invention;

FIGS. 10A and 10B are explanatory diagrams for describing a reading operation without having a coordinate computing function on the two-dimension code reader of the automatic analysis device according to the first embodiment of the present invention;

FIG. 11 is a configuration diagram illustrating an overall configuration of an automatic analysis device according to a second embodiment of the present invention;

FIG. 12 is a top view of an area near a reagent cool box of the automatic analysis device according to the second embodiment of the present invention;

FIG. 13 is an enlarged diagram of an area near a mounting portion of a two-dimension code reader of the automatic analysis device according to the second embodiment of the present invention;

FIG. 14 is a diagram illustrating an arrangement of reagent containers in the reagent cool box of the automatic analysis device according to the second embodiment of the present invention;

FIG. 15 is an explanatory diagram for describing a reading range of a two-dimension code label of an upper portion of the reagent container of the automatic analysis device according to the second embodiment of the present invention;

FIG. 16 is an explanatory diagram for describing types of codes to be read of the two-dimension code label of the upper portion of the reagent container of the automatic analysis device according to the second embodiment of the present invention; and

FIG. 17 is a diagram illustrating an example of read information by the two-dimension code reader of the automatic analysis device according to the second embodiment of the present invention.

### BEST MODE FOR CARRYING OUT THE INVENTION

Hereinafter, embodiments of the present invention will be described in detail with reference to the accompanying drawings. Note that components having the same function are denoted by the same reference symbols throughout the drawings for describing the embodiment, and the repetitive description thereof will be omitted.

### First Embodiment

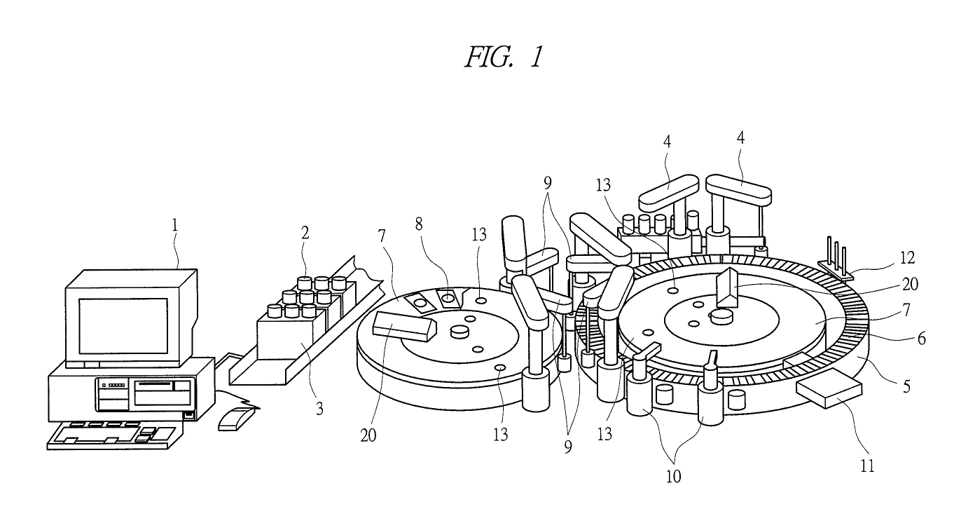

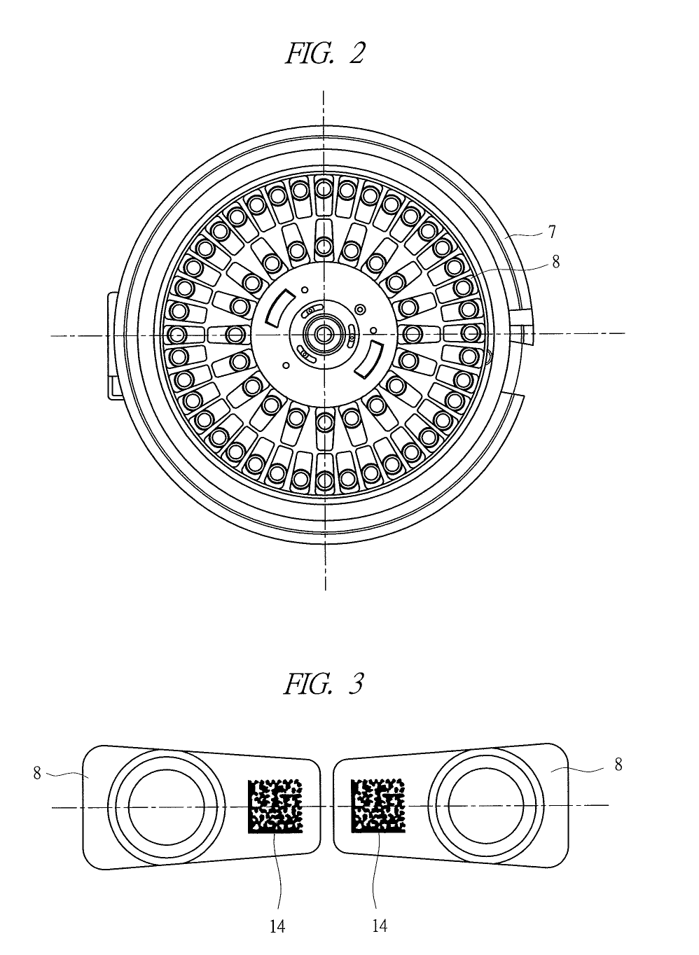

With reference to FIGS. 1 to 4, a configuration of an automatic analysis device according to a first embodiment of the present invention will be described. FIG. 1 is a configuration diagram illustrating an overall configuration of the automatic analysis device according to the first embodiment of the present invention, illustrating a cross section of a part of a cap of a reagent cool box so that a part of a plurality of reagent containers being kept cool is visible. FIG. 2 is a top view of the reagent cool box of the automatic analysis device according to the first embodiment of the present invention, illustrating a state in which the cap is removed. FIG. 3 is a diagram illustrating an arrangement of reagent containers of the automatic analysis device according to the first embodiment of the present invention, and FIG. 4 is a diagram illustrating an arrangement of a two-dimension code reader of the automatic analysis device according to the first embodiment of the present invention.

In FIG. 1, the automatic analysis device is composed of: an operation portion 1 performing various operations of the automatic analysis device; a sample container 2 containing a sample to be used in an analysis and having code information relating to the sample attached thereto; a conveying rack 3 for installing the sample container 2; a sample dispensing mechanism 4 dispensing a measurement liquid which is an analysis target from the sample container 2; a reaction disc 5 for installing reaction container 6; the reaction container 6 in which a measurement sample which is an analysis target and a reagent are reacted; a reagent cool box installing a reagent container 8; the reagent container 8 containing a reagent; a reagent dispensing mechanism 9 dispensing a sample which is an analysis target to the reaction container 6; an agitating mechanism 10 agitating an inside of the reaction container 6 in which the sample which is the analysis target and the reagent are dispensed; a photometer 11 being a detection mechanism detecting a state inside the reaction container 6 after agitation by the agitating mechanism 10; and a washing mechanism 12 washing inside the reaction container 6 after finishing an analysis.

In addition, a control device (not illustrated) saving information relating to the reagent, analyzing the sample which is the analysis target based on the information related to the reagent and a detection result from the photometer 11, and controlling the whole of the automatic analysis device is also provided. Note that the control device may be integrated with the operation portion 1.

The reagent cool box 7 keeps a plurality of the reagent containers 8 cool in reagent installing means, in which a reagent is filled, arranged on circumferences of an inner periphery and an outer periphery, and at least one opening 13 for sucking out the reagent from the reagent container 8 and a two-dimension code reader mounting portion 20 to which a two-dimension code reader is mounted are provided to the reagent cool box 7.

Also, as illustrated in FIGS. 2 and 3, in the reagent cool box 7, an installation position of the reagent container 8 of the inner periphery is arranged in a cyclic manner to be aligned in one straight line with respect to an installation position of the reagent container 8 of the outer periphery; and directions of the reagent container 8 on the inner periphery and the reagent containers 8 of the outer periphery are opposite so that two-dimension code labels 14 attached to upper portions of the reagent container 8 of the inner periphery and the reagent containers 8 of the outer periphery are arranged to be close.

In addition, as illustrated in FIG. 4, inside the two-dimension code reader mounting portion provided to the cap of the reagent cool box 7, for example, one two-dimension code reader 21 is mounted by a two-dimension code reader fixing plate 23, and an opening 22 is provided so that the two-dimension code label attached to the upper portion of the reagent container 8 can be read.

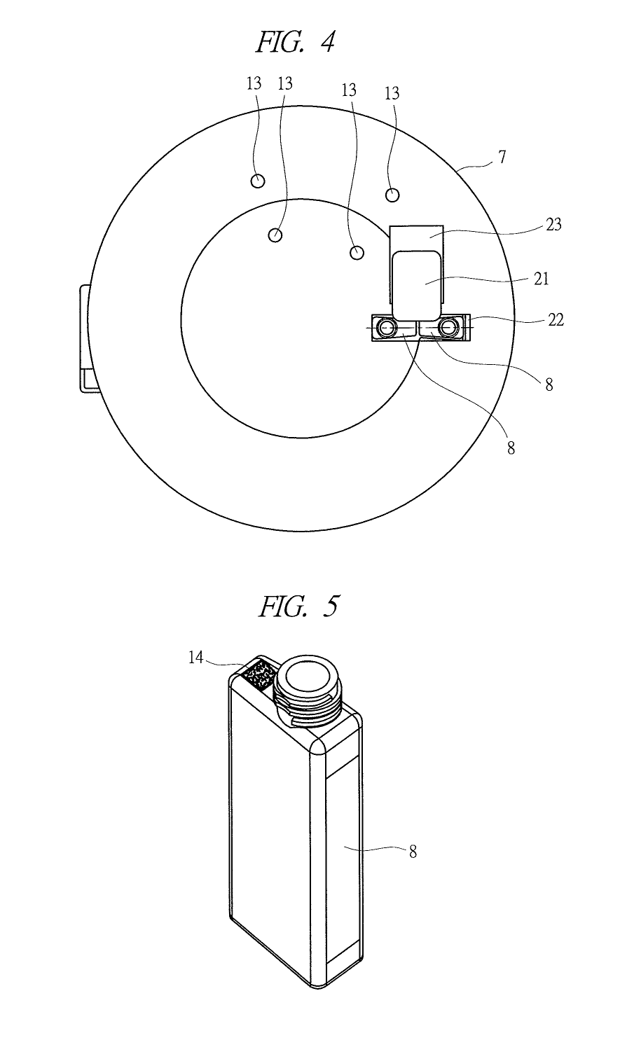

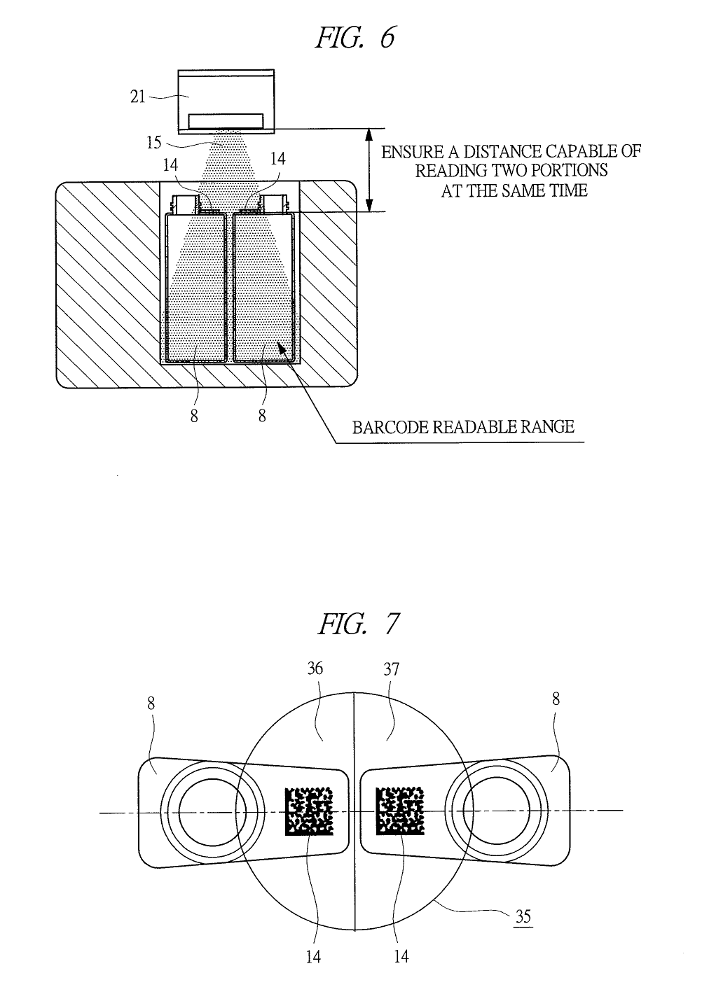

Next, with reference to FIG. 1 and FIGS. 5 to 7, operations of the automatic analysis device according to the first embodiment of the present invention will be described. FIG. 5 is a diagram illustrating an outer appearance of the reagent container of the automatic analysis device according to the first embodiment of the present invention, FIGS. 6 and 7 are explanatory diagrams for describing a reading range of the two-dimension code reader of the automatic analysis device according to the first embodiment of the present invention.

First, in the reagent cool box 7, the reagent containers 8 are previously arranged to the reagent installing means of the inner periphery and the outer periphery by an operator of the automatic analysis device.

And, according to instructions from the operation portion 1, the reagent container 8 to which the two-dimension code label 14, on which a two-dimension code is printed, is attached is moved to a lower portion of the two-dimension code reader 21 to be at a position for reading by the two-dimension code reader 21.

And, individual information per the reagent container 8 is acquired by the two-dimension code reader 21 installed to the cap of the reagent cool box 7.

Here, reading of the two-dimension code label 14 by the two-dimension code reader 21 will be described.

As illustrated in FIG. 5, the upper portion of the reagent container 8 has a flat surface portion, and the two-dimension code label 14 is attached to the flat surface portion. The code memorizing data of reagent information of the two-dimension code label 14 is, for example, DataMatrix, and specifications to be necessary in reading a code such as ensuring a quiet zone are ensured.

Also, the two-dimension code reader 21 is, as illustrated in FIG. 6, arranged at a position so that the two-dimension code labels 14 attached to both the reagent container 8 of the inner periphery and the reagent container 8 of the outer periphery are within an entire visual field 35 at the same time, i.e., one label for each of the reagent containers 8 on the inner periphery and outer periphery.

Here, as illustrated in FIG. 6, without arranging the reagent container 8 of the inside and the reagent container 8 of the outside in opposite directions, if the two-dimension code labels 14 attached to the reagent container 8 of the inside and the reagent container 8 of the outside are within a view field of one two-dimension code reader 21, the reagent container 8 of the inside and the reagent container 8 of the outside can be put in the same direction.

In addition, the two-dimension code reader 21 has a coordinate computing function for computing a coordinate (x, y) of a code recognized within the view field, and has a point of origin (0, 0) at, for example, the center of the view field. The reader divides the view field into two according to the coordinate information and performs reading in an order by applying a priority order so that codes can be discriminated even when there are a plurality of codes within the entire view field 35.

Further, the two-dimension code reader 21 can transmit position information in addition to ID information of the two-dimension code label 14, where the position information may be coordinate itself or data indicating a position such as “inner periphery side” or “outer periphery side”. Here, the arrangement of the two-dimension code reader 21 is at a position capable of dividing the view field into the inner periphery side and the outer periphery side.

Moreover, in the present embodiment, as illustrated in FIG. 7, the two-dimension code reader 21 divides the entire view field 35 into two, i.e., a view field 36 of the inner periphery side and a view field 37 of the outer periphery side according to the coordinate information.

Next, the two-dimension code reader 21 performs read from a view field in a previously-set order, and when the reading is succeeded, both of information of the two-dimension code label 14 and position information are transmitted. When the reading is not succeeded due to a lack of the reagent container 8 or other reasons, read error information is transmitted or nothing is done and then read in the next view field is performed. When reads of the inner periphery and outer periphery are finished, to perform read of the next reagent container 8, the inside of the reagent cool box 7 is rotated and read is performed in the same manner, reading all information items of the two-dimension code label 14 attached to the reagent container inside the reagent cool box 7, and then the information transmitted from the two-dimension code label 14 is stored in the control device etc.

Thereafter, the conveying rack 3 to which the sample container 2 containing a sample is installed is conveyed to an analyzing unit composed of the sample dispensing mechanism 4, the reaction disc 5, the reaction container 6, the reagent cool box 7, the reagent container 8, the reagent dispensing mechanism 9, the agitating mechanism 10, the photometer 11, and the washing mechanism 12.

As the sample conveyed to the analyzing unit is subjected to an analysis instructed from the operator 1, a measurement sample which is an analysis target inside the sample container 2 is sucked using the sample dispensing mechanism 4 and injected to the reaction container 6 installed to the reaction disc 5.

Also, based on information of the reagent container 8 previously acquired, the reagent container 8 installed inside the reagent cool box 7 is, to suck the predetermined reagent, moved to a position of the opening of the cap so that the reagent inside the reagent container 8 is sucked and injected to the reaction container 6 being put on the reaction disc 5.

The sample and reagent injected to the reaction container 6 are agitated by the agitating mechanism 10. Color of the chemical reaction of the agitation is measured by the photometer 11 which is a detection mechanism formed of a light-source lamp, a spectroscopic diffraction grating, and an optical detector, and an analysis is performed according to information from the photometer 11.

After the analysis, to analyze the next sample, the reaction container is washed by the washing mechanism 12. After sucking the sample for performing an analysis, the conveying rack 3 to which the sample container 2 is installed is conveyed from the analyzing unit.

As described in the foregoing, in the present embodiment, by installing the reagent container 8 as illustrated in FIG. 2, when reading the two-dimension code label 14 attached to the upper portion of the reagent containers 8 of the outer periphery by one two-dimension code reader 21, the reagent container 8 of the inner periphery is also arranged in a horizontally aligned manner, and thus it is possible to read also the two-dimension code label 14 attached to the upper portion of the reagent container 8 of the inner periphery as well as that of the outer periphery.

Therefore, as long as the two-dimension code reader 21 is disposed at a position capable of reading both the two-dimension code label 14 attached to the upper portions of the reagent container 8 of the inner periphery and the two-dimension code label 14 attached to the reagent container 8 of the outer periphery, by carrying out only a sequence of reading the two-dimension code label 14 attached to the upper portion of the reagent container 8 of the outer periphery, the two-dimension code label 14 attached to the upper portion of the reagent container 8 of the inner periphery can be read at the same time.

Also, by the two-dimension code reader 21, a coordinate (x, y) of a code recognized in the view field is computed and the view field is discriminated according to the coordinate, so that the information of the two-dimension code labels 14 attached to the reagent container 8 of the inner periphery and the reagent container 8 of the outer periphery is discriminated and recognized; therefore, it is possible to read information of all of the two-dimension code labels 14 attached to the reagent containers 8 by one two-dimension code reader 21.

Note that, while an example of arranging one reagent container 8 to each of the inner periphery and outer periphery in the reagent cool box 7 has been described in the present embodiment, it is considered possible to correspond to arranging two or more reagent containers 8 to the inner periphery and two or more reagent containers 8 to the outer periphery by increasing the number of divided view fields of the two-dimension code reader 21.

Also, while the view field is divided and added with a priority order in the foregoing description, this is aimed for shortening a time for code search, and thus the field division is not necessary when a code search is not necessary and the read can be performed until code recognition becomes unavailable while eliminating coordinates after finishing read in the view field.

In addition, while it has been described on the premise that the two-dimension code reader 21 can compute a coordinate of a code read from the two-dimension code label 14, if one two-dimension code reader 21 can read discriminating codes of a plurality of the two-dimension code labels 14, the coordinate computing function of code position is not always necessary in the two-dimension code reader 21, and, for example, codes of the plurality of two-dimension code labels 14 can be read with discriminating the codes by limiting the view field by view field limiting means or the like and referencing view field limitation information of the limited view field.

In addition, the two-dimension code labels 14 attached to the reagent container 8 of the inner periphery and the reagent container 8 of the outer periphery are not limited to those of the upper portions of the reagent containers 8, and may be attached to a side surface or a bottom surface of the reagent container 8, for example. In this case, an offset is provided from the surface to which the two-dimension code label 14 is attached to a position at which a necessary read view field can be ensured, and the two-dimension code reader 21 is arranged at the position. Then, according to a view field dividing or view field limiting means, codes are discriminated and read per periphery. When there are one or more inner peripheries and one or more outer peripheries, in the same manner, codes are discriminated and read per periphery according to a view field dividing or view field limiting means.

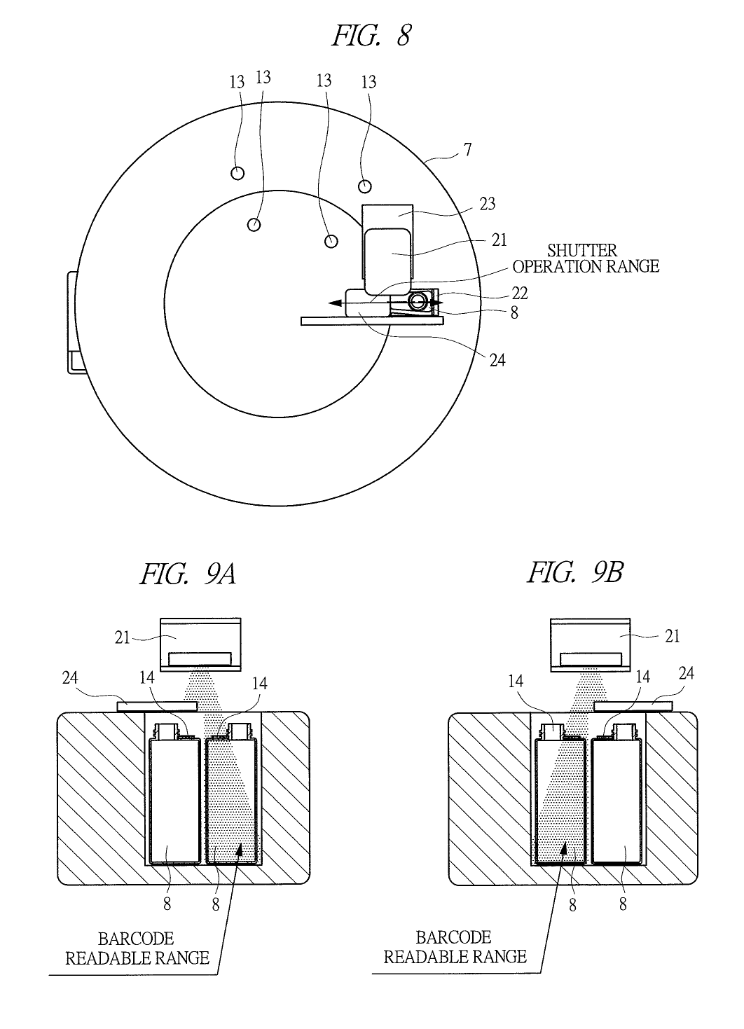

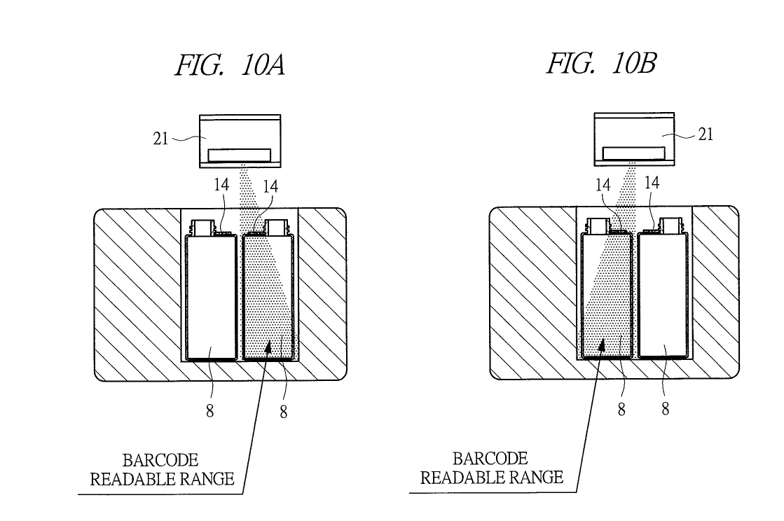

Here, with reference to FIGS. 8 to 10B, an example of read operation having no coordinate computing function on the two-dimension code reader of the automatic analysis device according to the first embodiment of the present invention. FIGS. 8 to 10B are explanatory diagrams for describing a read operation when there is no coordinate computing function on the two-dimension code reader of the automatic analysis device according to the first embodiment of the present invention, in which FIG. 8 and FIGS. 9A and 9B illustrate a situation of using opening/closing of a shutter for discriminating the inner periphery and outer periphery, FIG. 9A illustrating a state of selecting the outer periphery side, and FIG. 9B illustrating a state of selecting the inner periphery side. FIGS. 10A and 10B illustrate a situation of using a range of illumination for discriminating the inner periphery and outer periphery, FIG. 10A illustrating a state of selecting the outer periphery side and FIG. 10B illustrating a state of selecting the inner periphery side.

First, as illustrated in FIG. 8, the configuration may be such that, under the two-dimension code reader 21, a shutter 24, which is view field limiting means selecting the inner periphery or outer periphery and shielding the opening portion 22 of the cap of the reagent cool box 7, is provided, and the view fields of the inner periphery and outer periphery of the two-dimension code reader 21 are limited and read in an order.

When using the shutter 24, as illustrated in FIG. 9A, to read the two-dimension code label 14 attached to the reagent container 8 on the outer periphery side, the shutter 24 is moved to the inner periphery side so that the view field of the inner periphery side is limited. Also, as illustrated in FIG. 9B, to read the two-dimension code label 14 attached to the reagent container 8 on the inner periphery side, the shutter 24 is moved to the outer periphery side so that the view field of the outer periphery side is limited.

In this manner, the inner periphery and outer periphery can be discriminated according to position information of the shutter 24, and thus, even when the two-dimension code reader 21 does not have the coordinate computation function, the reagent container 8 of the inner periphery and the reagent container 8 of the outer periphery can be discriminated and information of the two-dimension code label 14 can be read.

In this case, assuming a situation in which the shutter 24 does not operate, for example, when a label on which an alarm code is written is attached on a surface of the shutter 24, the control device can recognize whether the shutter 24 has been operated or not upon read, and thus a sensor for shutter operation confirmation is not necessary.

Also, it is dark inside the reagent cool box 7 since the reagent cool box 7 is sealed, and thus illumination is necessary to read the code of the two-dimension code label 14.

Accordingly, as illustrated in FIGS. 10A and 10B, for example, the configuration is such that a range of illumination by LED light which is view field limiting means is divided to the inner periphery and outer periphery and by illuminating and reading them in an order, the view fields of the inner periphery and outer periphery are limited with the illumination and read in an order.

When using the illumination, as illustrated in FIG. 10A, to read the two-dimension code label 14 attached to the reagent container 8 on the outer periphery side, only the outer periphery side is illuminated with the illumination so that the view field of the inner periphery side is limited. Also, as illustrated in FIG. 10B, to read the two-dimension code label 14 attached to the reagent container 8 on the inner periphery side, only the inner periphery side is illuminated with the illumination so that the view field of the outer periphery side is limited.

In this manner, the inner periphery and outer periphery can be discriminated according to information of illuminating position of the illumination, and thus, even when the two-dimension code reader 21 does not have the coordinate computing function, the reagent container 8 of the inner periphery and the reagent container 8 of the outer periphery can be discriminated and information of the two-dimension code label 14 can be read.

In this case, assuming a situation in which the illumination is not turned on, a measure of providing a mechanism capable of determining whether the brightness of the illumination is sufficient or not, and so forth are considered. A function of detecting a dark state can be provided to the two-dimension code reader 21 itself, or confirmation of illumination can be performed by pasting a dummy label to one portion on a circumference of the reagent cool box 7 and determining whether the dummy label can be read or not.

Note that, while the example has been described in the example illustrated in FIGS. 10A and 10B about illumination from the inside of the two-dimension code reader 21, the inner periphery and outer periphery can be discriminated by illumination from the outside of the two-dimension code reader 21.

Also, using a function of trimming the view field range which the two-dimension code reader 21 has, the two-dimension code reader 21 is set so that only code information of one of the peripheries can be read to read code information of only one of the peripheries.

### Second Embodiment

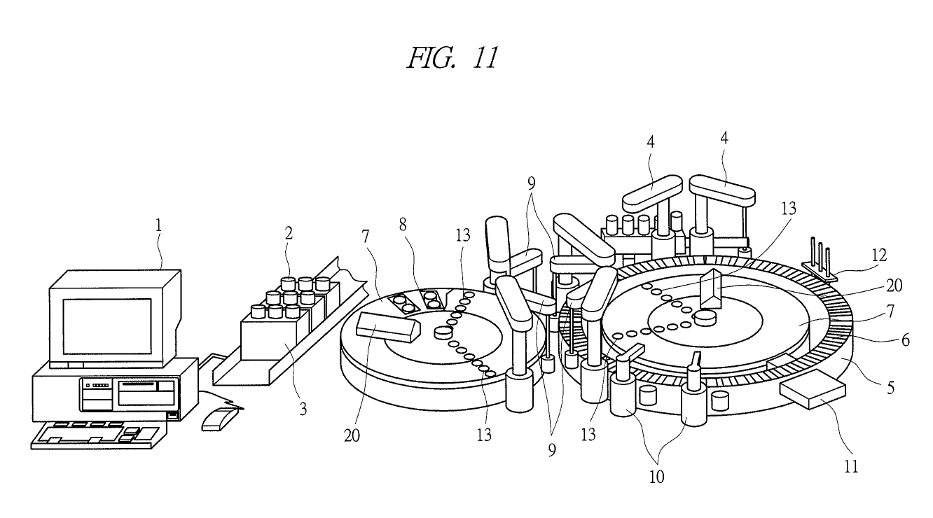

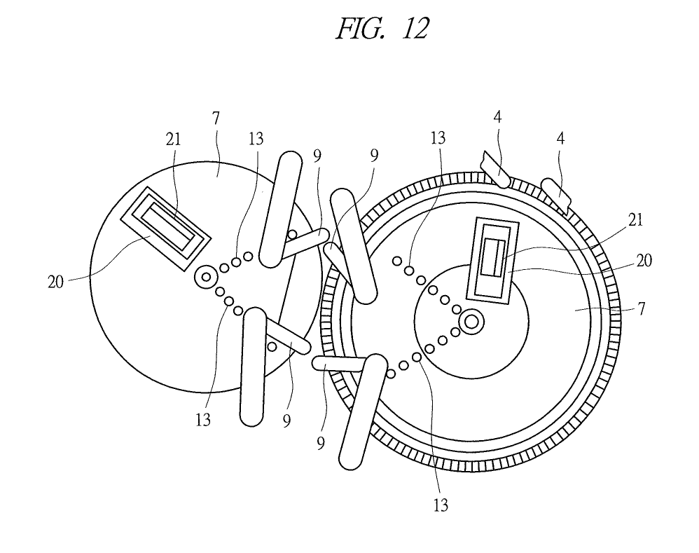

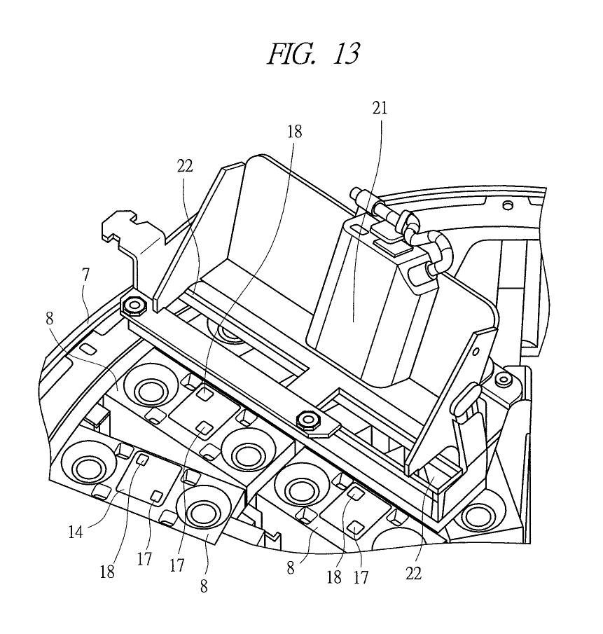

With reference to FIGS. 11 to 13, a configuration of an automatic analysis device according to a second embodiment will be described. FIG. 11 is a configuration diagram illustrating an overall configuration of the automatic analysis device according to the second embodiment of the present invention, in which a cross section of a part of a cap of a reagent cool box is expressed and apart of a plurality of reagent containers being kept cool is visible. FIG. 12 is a top view of an area near the reagent cool box of the automatic analysis device according to the second embodiment of the present invention, and FIG. 13 is an enlarged diagram of an area near a mounting portion of a two-dimension code reader of the automatic analysis device according to the second embodiment of the present invention.

In FIG. 11, the automatic analysis device is composed of: an operation portion 1 performing various operations of the automatic analysis device; a sample container 2 containing a sample to be used in an analysis and having code information relating to the sample attached thereto; a conveying rack 3 installing the sample container 2; a sample dispensing mechanism 4 dispensing a measurement liquid which is an analysis target from the sample container 2; a reaction disc 5 installing a reaction container 6; the reaction container 6 in which a measurement sample which is an analysis target and a reagent are reacted; a reagent cool box 7 installing a reagent container 8; the reagent container 8 containing a reagent; a reagent dispensing mechanism 9 dispensing a sample which is an analysis target to the reaction container 6; an agitating mechanism 10 agitating an inside of the reaction container 6 in which the sample which is the analysis target and the reagent are dispensed; a photometer 11 being a detection mechanism detecting a state inside the reaction container 6 after agitation by the agitating mechanism 10; and a washing mechanism 12 washing inside the reaction container 6 after finishing an analysis.

In addition, a control device (not illustrated) saving information relating to the reagent, analyzing the sample which is the analysis target based on the information related to the reagent and a detection result from the photometer 11, and controlling the whole of the automatic analysis device is also provided. Note that the control device may be integrated with the operation portion 1.

The reagent cool box 7 keeps a plurality of the reagent containers 8, in which a reagent is filled, arranged on circumferences of an inner periphery and an outer periphery cool, and at least one opening 13 for sucking the reagent from the reagent container 8 and a mounting portion of two-dimension code reader 20 to which a two-dimension code reader is mounted are provided.

In addition, as illustrated in FIG. 12, inside the mounting portion of two-dimension code reader 20 provided to a cap of the reagent cool box 7, one two-dimension code reader 21 is mounted.

Also, as illustrated in FIG. 13, inside the mounting portion of two-dimension code reader 20, openings 22 are provided so that a two-dimension code label 14 attached to an upper portion of the reagent container 8 can be read.

Moreover, to the two-dimension code label 14 attached to an upper portion of the reagent container 8, a DataMatrix code 17 and a QR code 18 are printed at diagonal positions.

Further, the two-dimension code reader 21 is arranged so that it is able to recognize a half area of each of the two-dimension code label 14 attached to the upper portion of the reagent container 8 of the inner periphery and the two-dimension code label 14 attached to the upper portion of the reagent container 8 of the outer periphery, respectively.

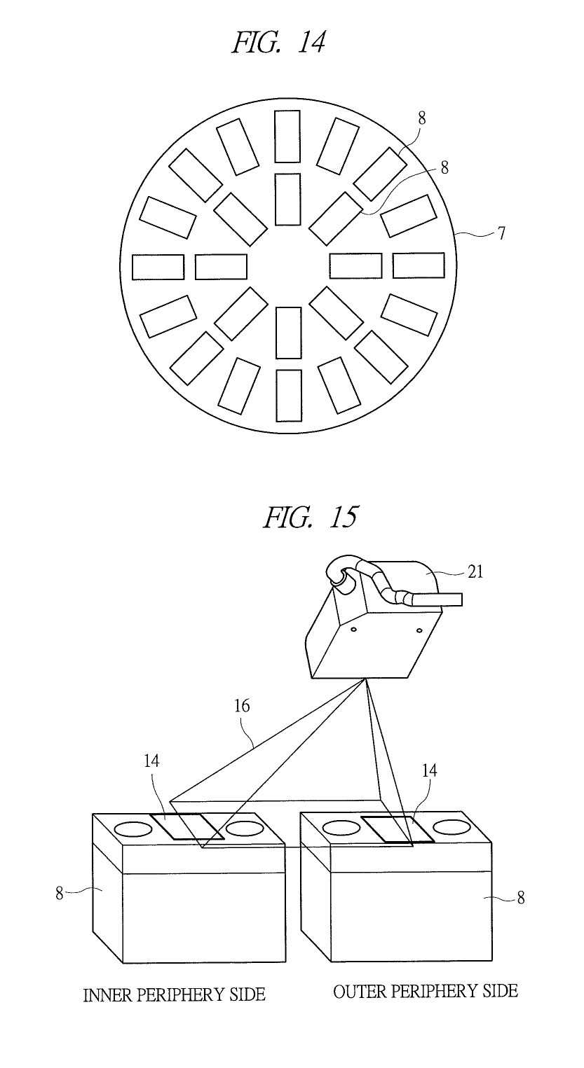

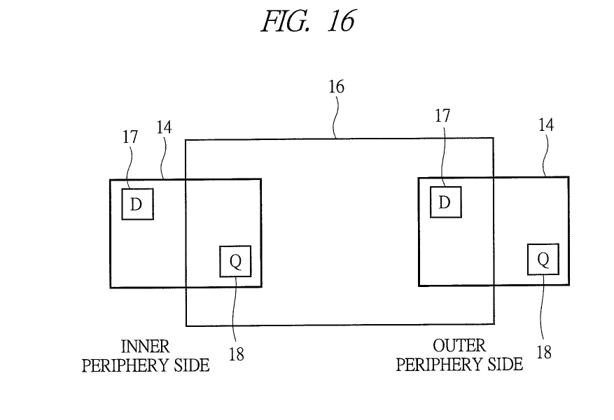

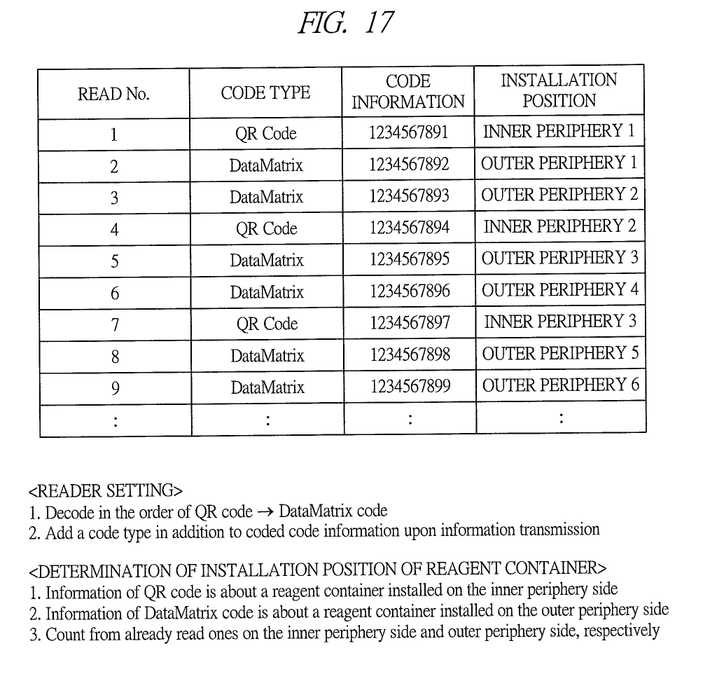

Next, with reference to FIGS. 11 and 14 to 17, operation of the automatic analysis device according to the second embodiment of the present invention will be described. FIG. 14 is a diagram illustrating an arrangement of the reagent containers inside the reagent cool box of the automatic analysis device according to the second embodiment of the present invention; FIG. 15 is an explanatory diagram for describing a reading range of a two-dimension code label on an upper portion of a reagent container of the automatic analysis device according to the second embodiment of the present invention; FIG. 16 is an explanatory diagram for describing code types to be read of the two-dimension code label on an upper portion of a reagent container of the automatic analysis device according to the second embodiment of the present invention; and FIG. 17 is a diagram illustrating an example of read information by the two-dimension code reader of the automatic analysis device according to the second embodiment of the present invention.

First, to the reagent cool box 7, the reagent containers 8 are previously disposed to reagent installing means of the inner periphery and outer periphery by an operator of the automatic analysis device.

And, according to instruction from the operation portion 1, the reagent container 8 to which the two-dimension code label 14, on which two-dimension codes of the DataMatrix 17 and QR code 18 are printed, is attached is moved to a lower portion of the two-dimension code reader 21 to be at a position for reading by the two-dimension code reader 21.

And, individual information per the reagent container 8 is acquired by the two-dimension code reader 21 installed to the cap of the reagent cool box 7.

Thereafter, the conveying rack 3 to which the sample container 2 containing a sample is installed is conveyed to an analyzing unit composed of the sample dispensing mechanism 4, the reaction disc 5, the reaction container 6, the reagent cool box 7, the reagent container 8, the reagent dispensing mechanism 9, the agitating mechanism 10, the photometer 11, and the washing mechanism 12.

As the sample conveyed to the analyzing unit is subjected to an analysis instructed from the operator 1, a measurement sample which is an analysis target inside the sample container 2 is sucked using the sample dispensing mechanism 4 and injected to the reaction container 6 installed to the reaction disc 5.

Also, based on information of the reagent container 8 previously acquired, the reagent container 8 installed inside the reagent cool box 7 is, to suck the predetermined reagent, moved to a position of the opening of the cap so that the reagent inside the reagent container 8 is sucked and injected to the reaction container 6 being put on the reaction disc 5.

The sample and reagent injected to the reaction container 6 are agitated by the agitating mechanism 10. Color of the chemical reaction of the agitation is measured by the photometer 11 which is a detection mechanism formed of a light-source lamp, a spectroscopic diffraction grating, and an optical detector, and an analysis is performed according to information from the photometer 11.

After the analysis, to analyze the next sample, the reaction container is washed by the washing mechanism 12. After sucking the sample for performing an analysis, the conveying rack 3 to which the sample container 2 is installed is conveyed from the analyzing unit.

Here, installation positions of the reagent containers 8 will be described. As illustrated in FIG. 14, inside the reagent cool box 7, an installation position of a reagent container of the inner periphery is arranged to be aligned with an installation position of a reagent container of the outer periphery in a cyclic manner and on a straight line.

Directions of alignment are in opposite directions in which faces in lateral directions of the reagent containers 8 of the inner periphery and outer periphery are facing to each other, and faces in longitudinal directions of the reagent containers 8 of the inner periphery and outer periphery are in directions on the same straight line to each other. By applying this way, when reading the two-dimension code label 14 attached to the upper portion of the reagent container 8 of the outer periphery by one two-dimension code reader 21, the reagent container 8 of the inner periphery is also horizontally aligned next in a cyclic manner, and thus it is possible to read the two-dimension code label 14 attached to the upper portion of the reagent container 8 of the inner periphery together with that of the outer periphery.

Therefore, when the two-dimension code reader 21 is provided at a position capable of reading both the two-dimension code label 14 attached to the upper portion of the reagent container 8 of the inner periphery and the two-dimension code label 14 attached to the upper portion of the reagent container 8 of the outer periphery, carrying out only a sequence of reading the two-dimension code label 14 attached to the upper portion of the reagent container 8 of the outer periphery enables reading of the two-dimension code label 14 attached to the upper portion of the reagent container 8 of the inner periphery also at the same time.

Next, positions of the reagent containers 8 of the inner periphery side and the outer periphery side at the reading position of the two-dimension code reader 21, positions of the two-dimension code labels 14 on which two-dimension codes are printed, and a read view field of the two-dimension code reader 21 will be described.

As illustrated in FIG. 15, the two-dimension code label 14 is attached to a flat surface of an upper portion of the reagent container 8. Also, to a top portion of the reagent container 8, one unit of the two-dimension code reader 21 of an imager system is mounted.

A read view field 16 exists in the two-dimension code reader 21 of imager system, and the two-dimension code reader 21 is provided such that the read view field is set across a half on the outer periphery side of the two-dimension code label 14 attached to the reagent container 8 of the inner periphery and a half on the inner periphery side of the two-dimension code label 14 attached to the reagent container on the outer periphery side.

For example, when the read view field is fixed, the position of the two-dimension code reader 21 is placed away from the two-dimension code label 14 and fixed, and, a view field range is adjusted if the read view field is variable by settings of the two-dimension code reader 21, thereby setting the read view field.

Next, a positional relationship of printing positions of the two-dimension codes on the two-dimension code label 14 and the read view field of the two-dimension code reader 21 will be described.

As illustrated in FIG. 16, on the two-dimension code label 14, two types of two-dimension codes of the DataMatrix code 17 and the QR code 18 are printed, where the DataMatrix code 17 and the QR code 18 are diagonally arranged on the two-dimension code label 14, and thus, the QR code 18 in the two-dimension code label 14 attached to the reagent container 8 on the inner periphery side and the DataMatrix code 17 in the two-dimension code label 14 attached to the reagent container 8 on the outer periphery side are positioned inside the read view field 16 of the two-dimension code reader 21.

In addition, the same information is encoded in the two types of codes of the DataMatrix code 17 and the QR code 18, and settings of the two-dimension code reader 21 are set such that both the DataMatrix code 17 and the QR code 18 can be previously read.

In this manner, the half on the outer periphery side of the two-dimension code label 14 of the inner periphery side is within the read view field range of the two-dimension code reader 21, and the QR code 18 is also within the read view field range, and thus it is possible to read by the two-dimension code reader 21.

In the same manner, the half on the inner periphery side of the two-dimension code label 14 of the outer periphery side is within the read view field range of the two-dimension code reader 21, and the DataMatrix code 17 is also within the read view field range, and thus it is possible to read by the two-dimension code reader 21.

In this manner, by reading, the reagent container 8 on the inner periphery side can be read as information regarding the reagent container 8 as the QR code 18, and the reagent container 8 on the outer periphery side can be read as information regarding the reagent container 8 as the DataMatrix code 17.

That is, in reading of the two-dimension code reader 21 at a place in which the reagent container 8 on the inner periphery and the reagent container 8 on the outer periphery are aligned, it is possible to determine that a reagent container is which of the reagent container 8 of the inner periphery and the reagent container 8 of the outer periphery depending on the code type of the DataMatrix code 17 and the QR code 18, and thus information items of the reagent container 8 of the inner periphery and the reagent container 8 of the outer periphery can be discriminated and read even when reading both the two-dimension code label 14 attached to an upper portion of the reagent container 8 of the inner periphery and the two-dimension code label 14 attached to an upper portion of the reagent container 8 of the outer periphery at the same time.

Next, determination of an installation place when reading the two-dimension code label 14 attached to an upper portion of the reagent container 8 by the two-dimension code reader 21 will be described.

As described above, the two-dimension code reader 21 is set so that both the DataMatrix code 17 and the QR code 18 can be read.

Further, when changing a reader setting of the two-dimension code reader 21 and, for example, reading the QR code 18 and the DataMatrix code 17 at once, information items are decoded in an order of QR code 18→DataMatrix code 17. Also, in addition to the decoded code information items, the code types are added upon transmission of read information.

Also, a sequence of read is such that: rotating the reagent container→stopping at the reading position→reader operation and read by inputting trigger→decoding information and transmitting the same to the operation portion 1 of the automatic analysis device and a central control portion etc.→rotating again so that a next reagent container 8 is at the reading position.

As described above, by constructing a time sequence in accordance with the number of installing the reagent containers 8 on the outer periphery side, the inner periphery can be also read together.

In addition, as the read is performed in the order of the QR code 18→the DataMatrix code 17, a sequence of regularity such that the QR code 18→the DataMatrix code 17→the DataMatrix code 17 appears. In this manner, when the regularity is missed, possibility of read miss of the two-dimension code reader 21 etc. can be detected.

And, by setting such that it is possible to identify (discriminate) that the QR code 18 is for the reagent container 8 of the inner periphery and the DataMatrix code 17 of for the reagent container 8 of the outer periphery, with discriminating the inner periphery and outer periphery in accordance with a sequential read rule, it is possible to register information of the reagent container 8 of the inner periphery in an order from an inner periphery 1 and information of the reagent container 8 of the outer periphery in an order from an outer periphery 1 as installation positions.

In the foregoing, while the invention made by the inventors of the present invention has been concretely described based on the embodiments, it is needless to say that the present invention is not limited to the foregoing embodiments and various modifications and alterations can be made within the scope of the present invention.

For example, while an example of reading the two-dimension code label 14 attached to the reagent container 8 has been described in the first embodiment, the two-dimension code label 14 to be a subject is applicable to not only the reagent container 8 but also things such as a specimen, accuracy management sample, or the like to which ID information or others is necessary to be provided.

In addition, while the DataMatrix code 17 has been used in the first embodiment, any code can be used as long as the code can be read by the two-dimension code reader 21.

Further, different types of codes or codes having different information amount in the inner periphery and outer periphery to each other.

Further, while an example of subjecting the reagent containers 8 of the inner periphery and outer periphery to a rotary drive integrally to each other has been described in the first embodiment, the inner periphery and the outer periphery can be subjected to an independent rotary drive. In this case, in a steady state, even when the reagent container 8 of the inner periphery and the reagent container 8 of the outer periphery are not arranged to be overlapped, it is sufficient when subjecting the inner periphery and outer periphery in a independent rotary drive and controlling such that the reagent container 8 of the inner periphery and the reagent container 8 of the outer periphery are overlapped at the reading position of the two-dimension code label 14 upon reading the two-dimension code labels 14 attached to the reagent containers 8 by the two-dimension code reader 21.

Further, while the QR code 18 and the DataMatrix code 17 have been used in the second embodiment, any codes can be used as long as the codes can be read by the two-dimension code reader 21 and are mutually different types of codes. For example, one-dimension barcode, a composite code of one-dimension and two-dimension, a two-dimension color code, etc. can be used.

Further, while the example of performing read in the order of the QR code 18→the DataMatrix code 17 has been described in the second embodiment, when the determination of regularity is not necessary, codes are simultaneously recognized and the information of the reagent containers 8 may be registered in an order with discriminating the reagent container 8 of the inner periphery and the reagent container 8 of the inner periphery.

Further, the two-dimension code label 14 has been attached to an upper portion of the reagent container 8 in the second embodiment, the two-dimension code label 14 can be attached to a place except for the upper portion of the reagent container 8 as long as the position is capable of reading the two-dimension code label 14 attached to the reagent container 8 of the inner periphery and the two-dimension code label 14 attached to the reagent container 8 of the outer periphery by one two-dimension code reader 21. Also, the codes are not necessarily arranged in a diagonal manner within the same label.

Further, the example of the reagent cool box 7 having reagent installing means of two peripheries of the inner periphery and outer periphery has been described in the second embodiment, reagent installing means for a plurality of peripheries may be provided. In this case, the two-dimension code labels 14 attached to the reagent container 8 installed to the reagent installing means of a plurality of peripheries are read by one two-dimension code reader 21 and code information items of different code types may be read with changing reading areas per periphery of the reagent installing means of the plurality of peripheries of the reagent cool box 7.

Further, the example of the reagent cool box 7 subjecting the reagent containers 8 of the inner periphery and outer periphery to a rotary drive being integrated with each other as illustrated in FIG. 14 has been described, the inner periphery and outer periphery may be subjected to an independent rotary drive. In this case, in a steady state, even when the reagent container 8 of the inner periphery and the reagent container 8 of the outer periphery are not arranged to be overlapped, it is sufficient when subjecting the inner periphery and outer periphery in a independent rotary drive and controlling such that the reagent container 8 of the inner periphery and the reagent container 8 of the outer periphery are overlapped at the reading position of the two-dimension code label 14 upon reading the two-dimension code labels 14 attached to the reagent containers 8 by the two-dimension code reader 21.

Moreover, the coordinate computing function, which has been described in the first embodiment, of computing a coordinate (x, y) of a recognized code in the view field among functions the two-dimension code reader 21 has can be used together. For example, the view field of the two-dimension code reader 21 is divided and position information and a target code are previously determined as a set such that the QR code 18 is read in a range of a divided view field on the inner periphery side, and the DataMatrix code 17 is read in a range of a divided view field on the outer periphery side. In code reading, when the position information and target code are consistent with those previously determined, it is recognized that reading is normally done, and, when the position information and target code are different from those previously determined, a user is notified that an error is detected such as upside-down placement of the container or others. Also, when determining the position information and target code as a set, the target code may not be included.

### INDUSTRIAL APPLICABILITY

The present invention relates to an automatic analysis device and widely applicable to a device which reads information such as reagent identification information added to a reagent container used in an analysis, and manages the same as information of a reagent, and so forth.

### EXPLANATION OF SYMBOLS

1 . . . Operation portion, 2 . . . Reagent container, 3 . . . Conveying rack, 4 . . . Sample dispensing mechanism, 5 . . . Reaction disc, 6 . . . Reaction container, 7 . . . Reagent cool box, 8 . . . Reagent container, 9 . . . Reagent dispensing mechanism, 10 . . . Agitating mechanism, 11 . . . Photometer, 12 . . . Washing mechanism, 13 . . . Opening, 14 . . . . Two-dimension code label, 16 . . . Reading view field, 17 . . . DataMatrix code, 18 . . . QR code, 20 . . . Mounting portion of two-dimension code reader, 21 . . . Two-dimension code reader, 22 . . . Opening, 23 . . . Fixing plate of two-dimension code reader, 24 . . . Shutter, 35 . . . Entire view field of two-dimension code reader, 36 . . . View field on inner periphery side of two-dimension code reader, and 37 . . . View field on outer periphery side of two-dimension code reader.

1. An automatic analysis device comprising:
a reagent container containing a reagent to be used in an analysis and attached with code information including information related to the reagent;
a reagent cool box including reagent installing means of a plurality of peripheries for installing a plurality of the reagent containers, the reagent installing means being provided at circumferences of concentric circles;
one code reader reading the code information attached to the reagent containers installed to the reagent installing means of a plurality of circumferences of the reagent cool box;
a reaction disc installing a plurality of reaction containers;
a sample dispensing mechanism dispensing a sample to be a target of analysis to the reaction container;
a reagent dispensing mechanism dispensing the reagent in the reagent container corresponding to the sample to be a target of analysis to the reaction container;
an agitating mechanism agitating inside the reaction container in which the sample to be a target of analysis is and the reagent are dispensed;
a detection mechanism detecting a state inside the reaction container after agitation by the agitating mechanism; and
a control device storing information related to the reagent and analyzing the sample to be a target of analysis based on the information related to the reagent and a detection result from the detection mechanism,
wherein, when reading the code information attached to the reagent container, the code reader computes position information of a position at which the code information is attached and outputs the position information with the read code information; and
the control device determines, based on the code information and the position information from the code reader, which periphery the code information of a reagent container installed to the reagent installing means of the periphery belongs to among the reagent installing means of the plurality of peripheries of the reagent cool box, and stores information related to the reagent together with information of an installation place of the reagent container.

2. The automatic analysis device according to claim 1,
wherein the code reader divides a view field per the reagent installing means of the plurality of circumferences of the reagent cool box, adds a priority order to the plurality of view fields, and reads the code information attached to the reagent container in the priority order.

3. The automatic analysis device according to claim 2,
wherein the code reader reads a plurality of items of the code information existing in the divided view fields and outputs the position information together with the read code information; and
the control device distinguishes and recognizes the plurality of items of the code information in the divided view fields based on the code information from the code reader, the position information, and the information of view fields.

4. An automatic analysis device comprising:
a reagent container containing a reagent to be used in an analysis and attached with code information including information related to the reagent;
a reagent cool box including reagent installing means of a plurality of peripheries for installing a plurality of the reagent containers, the reagent installing means being provided at circumferences of concentric circles;
one code reader reading the code information attached to the reagent containers installed to the reagent installing means of a plurality of circumferences of the reagent cool box;
a view field limiting means limiting a view field of the code reader per the reagent installing means of the plurality of circumferences of the reagent cool box;
a reaction disc installing a plurality of reaction containers;
a sample dispensing mechanism dispensing a sample to be a target of analysis to the reaction container;
a reagent dispensing mechanism dispensing the reagent in the reagent container corresponding to the sample to be a target of analysis to the reaction container;
an agitating mechanism agitating inside the reaction container in which the sample to be a target of analysis is and the reagent are dispensed;
a detection mechanism detecting a state inside the reaction container after agitation by the agitating mechanism; and
a control device storing information related to the reagent and analyzing the sample to be a target of analysis based on the information related to the reagent and a detection result from the detection mechanism,
wherein, when reading the code information attached to the reagent container, the code reader computes position information of a position at which the code information is attached and outputs the position information with the read code information; and
the control device determines, based on the code information from the code reader and view field limitation information from the view field limiting means, which periphery the code information of a reagent container installed to the reagent installing means of the periphery belongs to among the reagent installing means of the plurality of peripheries of the cool box, and stores information related to the reagent together with information of an installation place of the reagent container.

5. The automatic analysis device according to claim 4,
wherein the view field limiting means is a shutter disposed between the code reader and the reagent container; and
the view field limitation information is position information of the shutter.

6. The automatic analysis device according to claim 5,
wherein a dummy of the code information is attached to the shutter on the code reader side; and
the control device confirms operation of the shutter based on the code information of the dummy from the code reader and the position information of the shutter.

7. The automatic analysis device according to claim 4,
wherein the view field limiting means is an illumination irradiated for reading the code information by the code reader; and
the view field limiting information is irradiating position information of the illumination.

8. The automatic analysis device according to claim 7,
wherein a dummy of the code information is attached in the reagent cool box; and
the control device confirms brightness of the illumination based on the dummy of the code information from the code reader.

9. An automatic analysis device comprising:
a reagent container containing a reagent to be used in an analysis and attached with code information items of two or more different code types including information related to the reagent;
a reagent cool box including reagent installing means of a plurality of peripheries for installing a plurality of the reagent containers, the reagent installing means being provided at circumferences of concentric circles;
one code reader reading the code information attached to the reagent containers installed to the reagent installing means of a plurality of circumferences of the reagent cool box;
a reaction disc installing a plurality of reaction containers;
a sample dispensing mechanism dispensing a sample to be a target of analysis to the reaction container;
a reagent dispensing mechanism dispensing the reagent in the reagent container corresponding to the sample to be a target of analysis to the reaction container;
an agitating mechanism agitating inside the reaction container in which the sample to be a target of analysis is and the reagent are dispensed;
a detection mechanism detecting a state inside the reaction container after agitation by the agitating mechanism; and
a control device storing information related to the reagent and analyzing the sample to be a target of analysis based on the information related to the reagent and a detection result from the detection mechanism,
wherein, when reading the code information attached to the reagent container, the code reader reads code information items of different code types per each circumference of the reagent installing means of the plurality of circumferences of the reagent cool box, and outputs information of the code types together with the read code information; and
the control device determines, based on the code information and information of the code types from the code reader, which periphery the code information of a reagent container installed to the reagent installing means of the periphery belongs to among the reagent installing means of the plurality of peripheries of the reagent cool box, and stores information related to the reagent together with information of an installation place of the reagent container.

10. The automatic analysis device according to claim 9,
wherein the code reader reads code information items attached to the reagent containers of respective reagent installing means of the plurality of circumferences of the reagent cool box at the same time.

11. The automatic analysis device according to claim 10,
wherein the reagent containers installed to the reagent installing means of the plurality of circumferences of the reagent cool box are installed such that the reagent containers installed to the reagent installing means of the most outer periphery of the reagent cool box is aligned with the other reagent containers in one line directed to a center of the reagent cool box.

12. The automatic analysis device according to claim 11,
wherein, when reading the code information items attached to the reagent containers of respective circumferences of the reagent installing means of the plurality of circumferences of the reagent cool box, the code reader adds a priority order per the code type and reads the code information in accordance with the priority order, and outputs the read code information.

13. The automatic analysis device according to claim 12,
wherein the control device detects a read error of the code reader based on the code information items read in accordance with the priority order output from the code reader.
